(19) United States
(12) Patent Application Publication
(10) Pub. No.: US 2012/0109248 A1
(43) Pub. Date: May 3, 2012

(54) BATTERY DISCHARGE MEASUREMENT DEVICE AND METHOD

(76) Inventors: Therese Danielsson, Uppsala (SE); Johan Svahn, Bromma (SE); Marie Herstedt, Stockholm (SE); Mattias Tullberg, Uppsala (SE); Richard Williamson, Santa Monica, CA (US)

(21) Appl. No.: 13/383,249

(22) PCT Filed: Jul. 10, 2009
(86) PCT No.: PCT/SE2009/000363

§ 371 Date: Jan. 10, 2012

(51) Int. Cl. A61N 1/37; G06F 19/00; G01R 31/36

(52) U.S. Cl. 607/29; 702/63

(57) ABSTRACT

A battery discharge measurement device for determining the state of discharge for a battery has a battery voltage measurement unit adapted to measure and store a battery voltage and a battery usage activity detector for detecting a predefined battery usage activity draining current from the battery and triggering a voltage recovery period. A processor unit is provided for estimating a battery voltage of the battery during the voltage recovery period based on the measured and stored battery voltage and the number of predefined battery usage activities detected since the battery voltage measurement unit measured the battery voltage of the battery.

### BACKGROUND OF THE INVENTION

1. Field of the Invention

The present invention relates to a device and to a method for determining the state of discharge for a battery that are particularly suited for use in an implantable medical device, but the invention is generally useful in any application in which a determination of the state of discharge of a battery is needed.

2. Description of the Prior Art

At present, a wide variety of implantable medical devices (IMDs) are commercially available for clinical implantation that are programmable in a variety of operating modes and are interrogatable using high-speed wireless telemetry transmissions, e.g. radio frequency (RF) telemetry transmission. Such medical devices include implantable cardiac pacemakers, cardioverter/defibrillators, cardiomyostimulators, pacemaker/cardioverter/defibrillators, drug delivery systems, cardiac and other physiologic monitors, electrical stimulators including nerve and muscle stimulators, deep brain stimulators, and cochlear implants, and heart assist devices or pumps etc. Most such IMDs comprise electronic circuitry and an IMD battery that provides power to the electronic circuitry and that depletes in energy over time. Therefore, it is necessary to monitor the state of battery in such IMDs so that the IMD can be replaced before the battery depletes to a state that renders the IMD inoperable.

QHR (Q High Rate) batteries, which is based upon a combination of two cathode materials; CFₓ (Carbon Monofluoride) and SVO (Silver Vanadium Oxide), are being introduced in implantable cardioverter/defibrillator and tachycardia devices to replace the presently used SVO batteries to increase longevity and enable HV stable charge times. QHR battery is a high-performing, high-rate battery especially designed for the mentioned medical applications. Compared to traditional high-rate cells the QHR cell has superior deliverable energy density, lower internal resistance, higher current pulse capability and exceptional discharge stability.

Together with an industry-standard lithium anode, the QHR cell combines the high-power advantage of SVO with the exceptional discharge stability of CFₓ in a laminated plate cathode design, with multiple plate design flexibility. An SVO/CFₓ parallel cell design within the same casing is disclosed e.g. in U.S. Pat. No. 6,926,991. The energy-dense CFₓ enables long cell life at low discharge rates, while SVO provides intermittent, high-rate current application upon demand for therapy application, resulting in a cathode system that maximizes device performance.

For SVO batteries, estimates of the remaining longevity to elective replacement indication (ERI) and end of service (EOS) have been based upon battery voltage measurements in the device.

However, for QHR batteries, use of wireless telemetry, e.g. RF-telemetry, or charging of the high voltage (HV) capacitors, affect the battery voltage for longer time after the high current use. For charging of the HV capacitors, which has the largest impact, the battery voltage may be affected for extended periods which may exceed 20 days after the charge. During this time period the voltage first recovers to the value characteristic of its state of discharge, and may then also during a transient period continue to increase to higher than the expected value. The whole period is herein denoted voltage recovery period or time. During the voltage recovery period, real time measurement of the battery voltage cannot be used for correctly assessing the remaining longevity. Further, during the voltage recovery time ERI or EOS cannot be triggered on the measured battery voltage.

The recovery period duration depends e.g. on the amount of discharged capacity and the amount of high current used. The latter can for example be the number of HV charges.

U.S. Pat. No. 6,671,552 relates to a system and method for determining remaining battery life for an implantable medical device. The battery may include a combination of silver vanadium oxide and CFₓ. The estimates of the remaining life estimates are derived by periodically measuring battery voltage, and estimating the estimated past current drain of the IMD comprising an average of the sum of the quiescent current drain and therapy delivery current drain, and determining the estimated remaining longevity from the measured voltage and the estimated past current drain.

### SUMMARY OF THE INVENTION

An object of the present invention is to enable an improved estimation of the state of discharge of a battery during specified battery usage activities, and in particular for QHR batteries used in implantable medical devices, e.g. cardioverters and defibrillators in order to enable ERI detection during voltage recovery periods and also to obtain estimates of the remaining longevity to ERI.

According to the present invention, a voltage subtraction device and method is used to trigger ERI and to obtain estimates of the remaining longevity during the voltage recovery times in which real time measurements of the battery voltage cannot be used.

In a battery voltage region before ERI, battery voltage determinations made during the voltage recovery times will present a voltage estimated according to the present invention.

Generally this voltage is estimated as: The most recent valid battery voltage measurement value (i.e. not measured during the recovery time period) subtracted by X millivolts per battery usage activity that has occurred since the valid measurement was taken. This calculated battery voltage is then used for e.g. ERI triggering or for conservative estimates of remaining longevity.

The factor X may be chosen to correspond to the expected voltage decrease caused by both the capacity used by the battery usage activity and the pacing and sensing capacity consumption during the voltage recovery time, and the risk of late ERI triggering is then substantially decreased.

An estimated voltage is presented at times when a real time battery voltage measurement gives invalid readings.

Thus, the capacity used by both HV charging and pacing and sensing during its associated voltage recovery time may be translated to a decrease in battery voltage as a factor of X mV per HV charge.

In another embodiment the capacity used by HV charging only is determined as one battery usage activity, which is translated into a decrease in battery voltage per charge. The actual capacity used by sensing and pacing during the recovery period is another battery usage activity and is measured to provide an exact measure of battery voltage decrease during said recovery period.

For a period of the battery discharge curve where it is most likely to reach ERI and the capability to alert is most important, the battery voltage can be approximated to a linear function for the discharge capacity. This function is used to determine the factor of X mV per battery usage activity.

The device and method according to the present invention is simple and easy to implement, and designed and optimized to be effective with regards to the capacity and possibilities of a typical micro controller in an implantable device.

### BRIEF DESCRIPTION OF THE DRAWINGS

FIG. 1 is a schematic block diagram illustrating the present invention.

FIG. 2 is a graph illustrating the approximately linear relationship between the battery voltage and discharge capacity in the U_{threshold} to ERI region.

FIGS. 3-5 are graphs illustrating various aspects of the present invention.

FIG. 6 is a flow diagram illustrating the method of the present invention.

FIG. 7 is a flow diagram illustrating an embodiment of the method of the present invention.

### DESCRIPTION OF THE PREFERRED EMBODIMENTS

The invention will now be described in detail with references to the appended drawings.

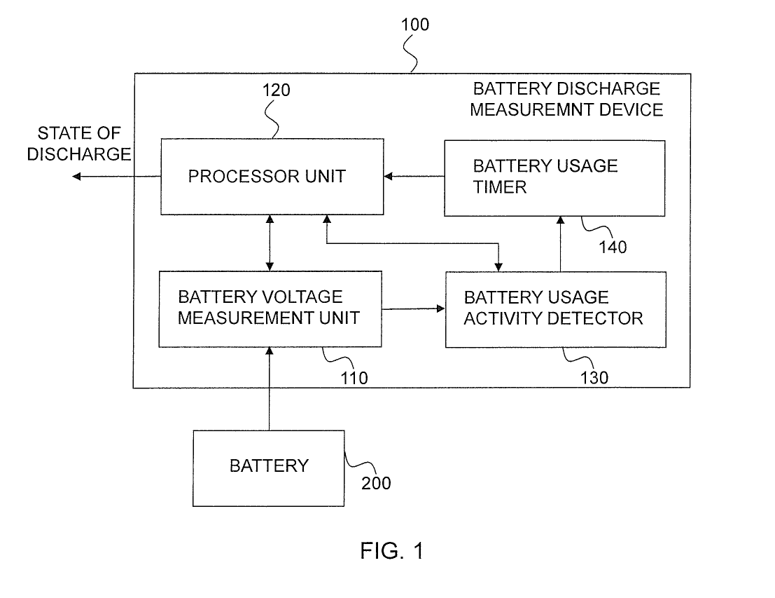

With reference to FIG. 1 the present invention relates to a battery discharge measurement device 100 for determining the state of discharge for a battery 200, preferably a QHR battery 200, but the present invention is also applicable for other battery types.

The device 100 includes a battery voltage measurement unit 110 adapted to measure and store a battery voltage, and a processor unit 120 connected to said battery voltage measurement unit 110.

The device 100 further includes a battery usage activity detector 130 connected to said units 110, 120 and adapted to detect predefined battery usage activities draining current of the battery 200. In a typical embodiment, such a predefined battery usage activity causes a voltage recovery period or time for the battery 200. During this voltage recovery period direct voltage measurements conducted by the battery voltage measurement unit 110 cannot be used for correctly assessing the remaining longevity of the battery 200. The present embodiments solve this problem through the operation of the battery usage activity detector 130. Thus, this battery usage activity detector 130 detects and calculates the number of predefined battery usage activities that has occurred since the battery voltage measurement unit 110 determined a valid battery measurement voltage. The processor unit 120 uses the this valid battery measurement voltage determined and stored by the battery voltage measurement unit 110 together with information of the calculated number of predefined battery usage activities as determined by the battery usage activity detector 130.

This means that during a voltage recovery period the battery voltage previously measured by the battery voltage measurement unit prior the voltage recovery period is modified with a factor related to the battery usage activity detected by the battery usage activity detector 130. This modification of the valid battery voltage allows correct estimation of battery voltage even during voltage recovery periods for the battery 200.

In a particular embodiment, the battery usage activity detector 130 process predefined battery usage activities and to generate a battery usage activity signal in response of detected and processed usage activities. The signal is applied to at least one battery usage timer 140 resulting in the timer 140 being started and set to run for a specified duration related to the battery usage activity.

There are different types of battery activities. Each type of battery usage activity is given an index number i. Xᵢ represents the battery voltage decrease caused by one battery usage activity with index number i. Nᵢ represent the number of battery activities with index number i that has occurred.

The battery voltage obtained, by the battery voltage measurement unit 110, when no battery usage timer is running is a valid battery measurement voltage U_{valid}.

In the case a battery usage timer is running an estimated battery voltage U_{estimated} is estimated, by the processor unit 120 as:

$${U}_{estimated} = {U}_{valid} - {\sum }_{i = 1}^{activities} {N}_{i} {X}_{i}$$

where
Nᵢ is the number of battery usage activities with index number i,
Xᵢ is a factor related to the battery usage activity with index number i, i.e. representing the battery drain caused by the battery usage activity.

A summation is made for activities of different types having index no. 1,2,3 . . . to determine the voltage U_{estimated} following a battery usage activity. Thus, in order to determine the estimated battery voltage U_{estimated} the sum of factors representing all battery usage activities that occur during the period when the timer 140 is running is subtracted from the valid battery voltage.

It should be noted that the sum may relate to the same or different battery usage activities, i.e. different activities results in different Xs. Thus it is observed that the formula above may be used in sequence if a battery usage activity occurs while the battery usage timer 140 is running. In such a case the current U_{estimated} is inserted as U_{valid} in the formula above when a new U_{estimated} is estimated to account for the latest battery usage activity. The parameter Xᵢ in the equation is determined using one or many of the following, non-exhaustive, list of inputs:
The expected battery variation
The expected current load variation
The estimated capacity per battery usage activity
The estimated capacity used in recovery period

The battery usage activities may thus be any high current use of the battery 200. For example, but not limited to, HV charging, telemetry, and antitachy pacing.

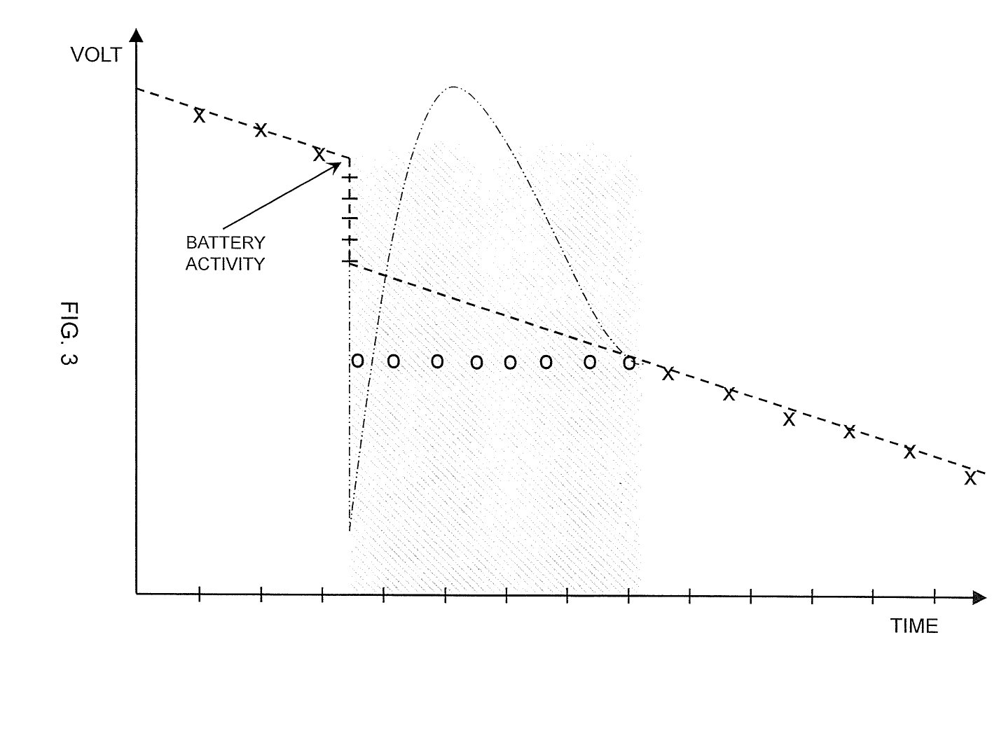

With references to the graphs in FIGS. 3-5 various aspects of the present invention will now be discussed. In the graphs the y-axis represents the battery voltage in volt and the x-axis represents the time (e.g. in days or hours).

The theoretical expected battery voltage is denoted by a dashed line, the assumed probable battery voltage behaviour is denoted by a dashed-dotted line, the valid battery voltage measurements are denoted by “X”, the calculated battery voltage values are denoted by “O”.

The dashed area represents the time when the battery usage timer is running.

With reference to FIG. 3, battery usage activity (i) occurs Nᵢ number of times (in this case five) which virtually would drain the battery to a lower voltage instantaneously (see the leap in the dashed line). However, the nature of the battery and its chemistry is not such that the voltage of the battery immediately after a high current drain battery usage activity does not correspond to the new actual remaining capacity. Instead, during a period after a battery usage activity like this, the voltage behaves in a way (dashed-dotted line) that is different from the known voltage-capacity relationship. This prevents temporarily the voltage to be used to determine remaining capacity.

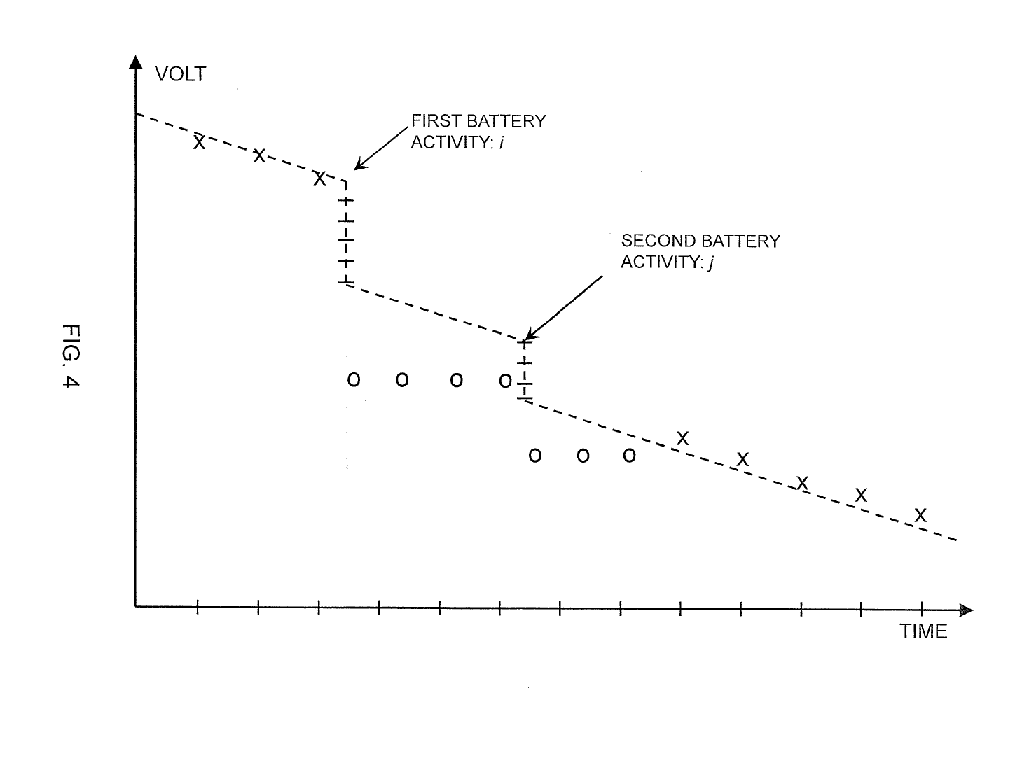

There is a controlled period, see dashed area, during which we cannot get a valid and reliable voltage measurement from the battery. During this voltage recovery period the voltage is estimated instead of being measured. The voltage is preferably estimated according to the formula above. Thus, the latest known valid value is used as a starting point. The Xᵢ factor which is individual for each type of battery usage activity is multiplied by the number of times it occurred (in this case five). If two or more battery usage activities occur simultaneously or within the timeframe of between two consecutive measurements, their negative contributions to the expected voltage are all summed together (as the situation is in FIG. 4). In FIG. 3 we only have one battery usage activity.

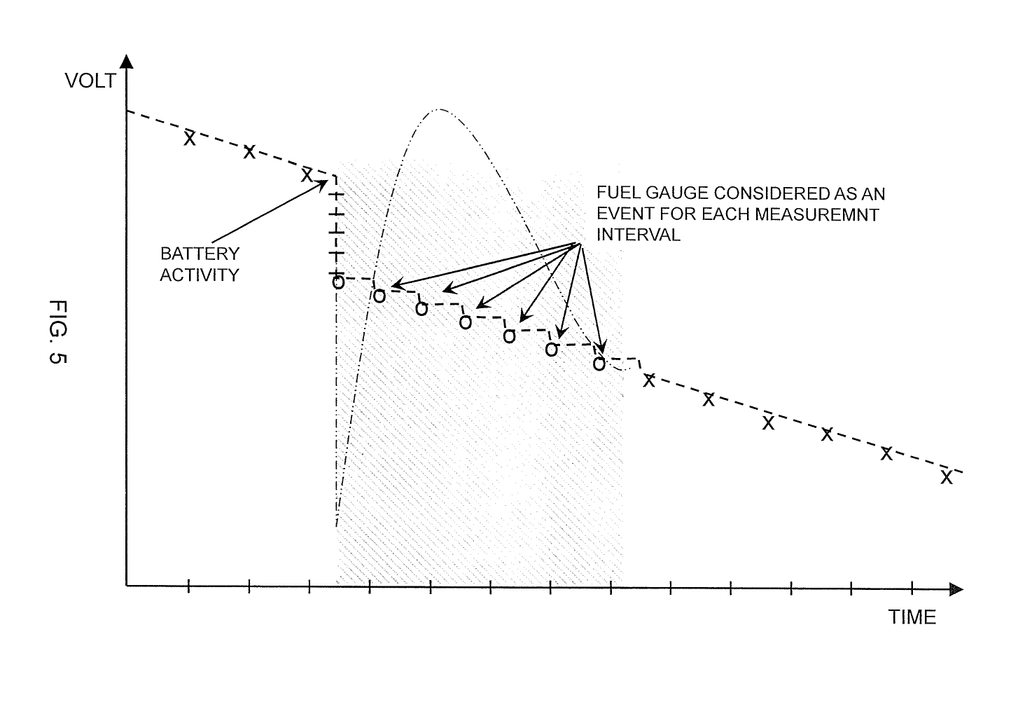

There are two options for deriving the Xᵢ factor of a battery usage activity. It can either be deducted so that the expected normal current drain during the following uncertain time period is accounted for, which is the case in FIG. 3 (this explains why the calculated battery voltage becomes and remains the voltage expected at the end of the time period). Alternatively, it can represent just the specific battery usage activity and not take into account the normal current drain. In the latter case the actual current drain measured by a fuel gauge or other means can be represented by a battery usage activity and thereby have its own subtraction factor Xᵢ. This is further discussed in relation to FIG. 5.

In FIG. 4 an initial battery usage activity, denoted i, has triggered a controlled period, i.e. started a timer. However, in this case another battery usage activity, denoted j, occurs before the first time period has elapsed. The second battery usage activity will cause the timer period to be updated (elongated) and the battery voltage estimated will include the subtraction factor for the second battery usage activity.

Described more in detail, the timer is started by the first battery usage activity, one or many battery usage activities, and an estimated battery voltage is calculated using the above equation, i.e. the last valid battery voltage is decreased by the sum of voltage values from all battery usage activities. As seen from the graph the estimated voltage value is well below the expected battery voltage (the dashed line), the reason is that also a safety margin is included when the voltage value of a battery usage activity is determined. The expected battery voltage is the battery voltage the device would measure if voltage recovery was instantaneous. Moreover, the expected battery voltage would also follow the known voltage-depleted capacity curve. Later, a further battery usage activity (a second battery usage activity), occurs. As the timer still is running, also the voltage values representing this second battery usage activity is decreased from the calculated voltage value. Probably this second battery usage activity also influences the duration of the timer period such that it is increased.

The situation shown in FIG. 5 is similar to the situation shown in FIG. 3.

However, in this case the updated fuel gauge value is considered a battery usage activity for every battery measurement made during the controlled period. The fuel gauge measurement will not update the controlled period.

The battery voltage values are e.g. calculated regularly, e.g. once each day, until the timer period has lapsed. Naturally, other calculation frequencies may be applied, e.g. a preset number of hours, e.g. every 10^{th} hour. The calculation may also be performed on demand, e.g. under the control of an external programming device, or as a consequence of a battery usage activity or other external influences, e.g. high temperature.

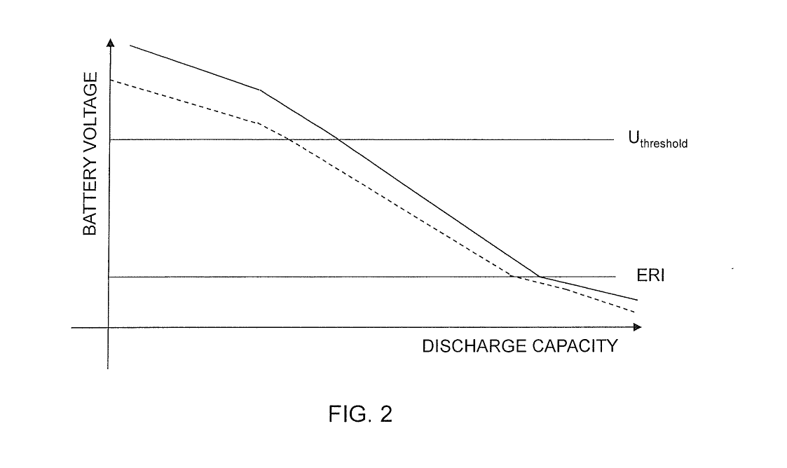

According to a preferred embodiment the battery voltage measurement unit is adapted to determine a linear battery discharge curve being a representation of the relationship between the battery voltage decrease per used mA hour capacity. This battery discharge curve is illustrated in FIG. 2. Preferably, a presumption to perform the calculation of the battery voltage is that the most recent valid battery measurement U_{valid} is less than a set threshold value U_{threshold}. U_{threshold} generally designates the start of the linear part of the expected battery discharge curve.

According to one embodiment the device comprises one battery usage timer commonly activated by all battery usage activities, or as an alternative the device comprises a number of battery usage timers, each related to a specified battery usage activity.

Thus, HV charging and wireless high-speed telemetry both may start (separate) recovery timers in the firmware. Voltage recovery time due to HV charging may be combined in one recovery timer or kept as two separate timers, depending on choice of implementation. Until the recovery timers expire all real time battery voltage measurements are marked invalid and prohibited for ERI or EOS triggering against nominal ERI or EOS references and for use by the programmer for longevity estimates. During recovery periods other voltage references for ERI or EOS triggering apply if battery voltages are measured during this period. Battery voltage measurements during recovery period may be made as a safety precaution to provide an early alarm in case of a premature battery depletion but this would not be a part of the normal ERI or EOS determination. Battery voltage measurements outside voltage recovery times are regarded as valid, i.e. are representative of the state of discharge.

As mentioned above the battery discharge measurement device is generally applicable but in particular useful to determine the state of discharge of a battery in an implantable medical device. The battery usage activity, when applied in an IMD, is preferably related to the charging of a HV capacitor, the use of high-speed telemetry, e.g. RF telemetry, and/or pacing and sensing energy consumption. As discussed above one or many timers may be arranged, wherein one of the timers is related to the charging of a HV capacitor and one of the timers is related to the use of RF telemetry.

The estimated U_{estimated} is used e.g. to estimate the remaining longevity to elective replacement indication (ERI) and/or end of service (EOS) for the battery.

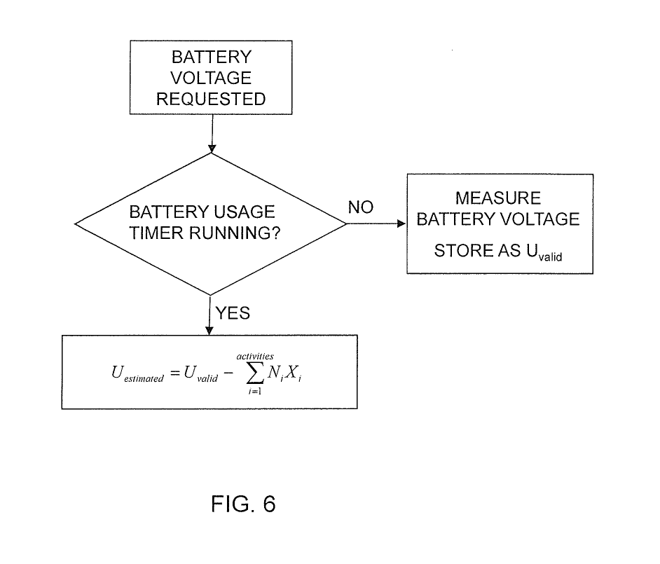

The present invention is also related to a method for determining the state of discharge for a battery. The method is schematically illustrated by the flow diagram of FIG. 6.

The method includes:
A) measuring and storing a battery voltage U_{valid} of said battery;
B) detecting at least one predefined battery usage activity; and
C) estimating a battery voltage U_{estimated} of said battery during a recovery period following a predefined battery usage activity based on the stored battery voltage U_{valid} and a number of predefined battery usage activities detected since measuring the battery voltage U_{valid}.
   In a particular embodiment the method also comprises:
D) processing predefined battery usage activities and generating a battery usage activity signal in response of detected and processed usage activities;
E) applying the battery usage activity signal to at least one battery usage timer resulting in that the timer is started and set to run for a specified duration related to the battery usage activity;
F) obtaining the battery voltage when no battery usage timer is running and denoting it a valid battery measurement voltage U_{valid}, and
G) estimating a battery voltage U_{estimated} when a battery usage timer is running, as:
   where
   Nᵢ represent the number of battery usage activities with index number i that has occurred,
   Xᵢ is a factor related to the battery usage activity with index number i, i.e. representing the battery drain caused by the battery usage activity with index number i.
   all different activities each having its own index are summed to provide a voltage decrease from the last measurement U_{valid}.

In the flow diagram in FIG. 6 a battery voltage request may be caused either by the normal battery status measurement (e.g. at regular intervals), or by a battery usage activity, e.g. HF charging, telemetry, etc.

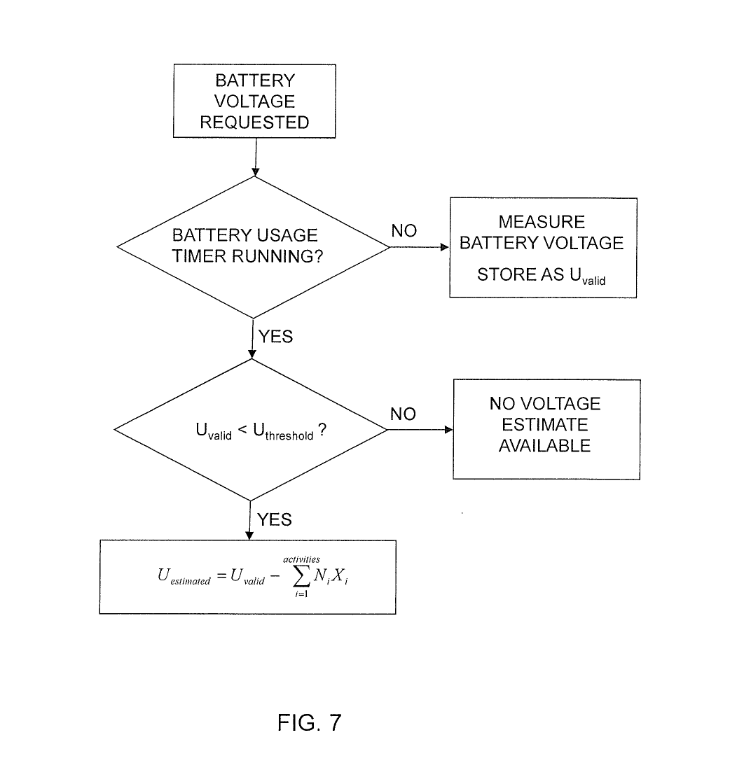

According to a further embodiment (see FIG. 7) the method further comprises determining a linear battery discharge curve being a representation of the relationship between the battery voltage decrease per used mA hour capacity. A presumption to perform the estimation of battery voltage is that the most recent valid battery measurement U_{valid} is less than a set threshold value U_{threshold}, (see FIG. 2). U_{threshold} generally designates the start of the linear part of the expected battery discharge curve. The method according to this embodiment is illustrated by the flow diagram in FIG. 7.

U_{threshold} is selected after which the battery discharge curve shows an approximately linear relationship between the millivolts battery decrease per used milliampere hour capacity until ERI is reached (see FIG. 2). When the battery voltage in a valid measurement is below U_{threshold} the method and device according to the present invention is used to estimate an expected battery voltage that is used for ERI triggering and longevity estimates for battery voltage measurements taken during recovery. This ensures ERI triggering even if e.g. HV charges occur frequently for a patient causing all real time battery measurements during a long time to be invalid. The U_{threshold} voltage is selected at a sufficiently long time before ERI to make it improbable to miss triggering of both U_{threshold} and ERI due to constantly being in a recovery period and hence only having invalid battery voltage measurements. The slope of the battery discharge curve may change during the battery life, thus the linear parts of the curve might change. Generally, the present invention is applicable to all linear parts of the discharge curve, i.e. the threshold values used to define the linear range may be variable.

The linear voltage subtraction method and device according to the present invention may for example be implemented as follows:

In the U_{threshold} to ERI range at all invalid battery voltage measurements a factor of X millivolts (mV) per HV charging that has occurred since the most recent valid battery measurement is subtracted from the voltage of the valid measurement. This calculated battery voltage is then used for ERI triggering, or by an external programmer device for longevity estimates. The factor X is chosen to correspond to the expected battery voltage decrease caused by both the capacity used by the HV charge, wireless telemetry and the pacing and sensing capacity consumption during the voltage recovery time. Choosing a factor of X mV per HV charge is possible thanks to the recognition of an approximately linear battery voltage to discharged capacity relationship in this U_{threshold} to ERI region (see FIG. 2). It is the latest valid battery voltage measurement in the range between U_{threshold} to ERI or the last U_{estimated} depending on which is most recent that will be used for the calculation.

Although the present invention is described in connection with ICDs and QHR batteries the voltage subtraction method and device according to the present invention may also be applicable for other types of devices and other battery types. The method and device are advantageous for any device and battery for which the battery must temporarily supply a higher current to support a temporary feature, causing the battery voltage to be affected for some time after the high current use. Estimations similar to those disclosed in the present application are useful for any period of battery discharge curve where the relationship between the battery voltage and the discharge capacity is approximately linear.

Although modifications and changes may be suggested by those skilled in the art, it is the intention of the inventors to embody within the patent warranted heron all changes and modifications as reasonably and properly come within the scope of their contribution to the art.

We claim as our invention:

1. A battery discharge measurement device for determining a state of discharge of a battery, comprising:
a battery voltage measurement unit adapted to measure and store a battery voltage;
a processor unit connected to said battery voltage measurement unit;
a battery usage activity detector connected to said processor unit and adapted to detect predefined battery usage activities; and
said processor unit being configured to estimate a battery voltage U_{estimated} based on said stored battery voltage and a number of predefined battery usage activities detected by said battery usage activity detector since said battery voltage measurement unit measured said battery voltage.

2. Battery discharge measurement device according to claim 1, wherein said predefined battery usage activities trigger a voltage recovery period, said battery voltage measurement unit is adapted to measure said battery voltage prior said voltage recovery period and said processor unit is configured to estimate said battery voltage U_{estimated} based on said stored battery voltage and said number of predefined battery usage activities during said voltage recovery period.

3. Battery discharge measurement device according to claim 1, wherein said battery usage activity detector is adapted to process said predefined battery usage activities and to generate a battery usage activity signal in response of detected and processed usage activities, said battery usage activity signal is applied to at least one battery usage timer resulting in that said battery usage timer being started and set to run for a specified duration related to said battery usage activity, said battery voltage measured, by said battery voltage measurement unit, when no battery usage timer is running is a valid battery measurement voltage U_{valid}, and wherein said processor unit is configured to estimate said battery voltage U_{estimated}, when a battery usage timer is running, as:
$${U}_{estimated} = {U}_{valid} - {\sum }_{i = 1}^{activities} {N}_{i} {X}_{i}$$
where
N₁ is the number of battery usage activities having index i,
Xᵢ is a factor related to said battery usage activity i, i.e. representing the battery drain caused by said battery usage activity, and
activities represent the number of different kinds of battery usage activities.

4. Battery discharge measurement device according to claim 3, wherein said battery voltage measurement unit is adapted to determine a linear battery discharge curve being a representation of a relationship between the battery voltage decrease per used ampere-hour capacity and that a presumption to perform said estimation is that the most recent valid battery measurement U_{valid} is less than a threshold value U_{threshold} representing the start of the linear part of the curve.

5. Battery discharge measurement device according to claim 3, wherein said battery discharge measurement device (100) comprises one battery usage timer commonly activated by all battery usage activities.

6. Battery discharge measurement device according to claim 3, wherein said battery discharge measurement device comprises a number of battery usage timers, each related to a specified battery usage activity.

7. Battery discharge measurement device according to claim 1, wherein said battery is a QHR battery.

8. (canceled)

9. Implantable medical device according to claim 18, wherein said device component that performs said battery usage activity is selected from the group consisting of a device that charges a high voltage (HV) capacitor, a wireless telemetry component, and a component of pacing and sensing circuitry.

10. Implantable medical device according to claim 18, wherein said battery usage activity detector is adapted to process said predefined battery usage activities and to generate a battery usage activity signal in response of detected and processed usage activities, said battery usage activity signal is applied to at least one battery usage timer, said at least one battery usage timer being related to charging of a HV capacitor.

11. Implantable medical device according to claim 18, wherein said battery usage activity detector is adapted to process said predefined battery usage activities and to generate a battery usage activity signal in response of detected and processed usage activities, said battery usage activity signal is applied to at least one battery usage timer, said at least one battery usage timer being related to the use of wireless telemetry.

12. Implantable medical device according to claim 18, wherein said estimated battery voltage U_{estimated} is made available in a form allowing estimation of a remaining longevity to elective replacement indication (ERI) and/or end of service (EOS) for said battery.

13. Method for determining a state of discharge for a battery, comprising
measuring and storing a battery voltage of said battery;
detecting at least one predefined battery usage activity;
estimating a battery voltage U_{estimated} of said battery based on said stored battery voltage and a number of predefined battery usage activities detected since measuring said battery voltage of said battery.

14. Method according to claim 13, wherein said predefined battery usage activities trigger a voltage recovery period for said battery, said measuring said battery voltage of said battery is performed prior said voltage recovery period and said estimating said battery voltage U_{estimated} is performed during said voltage recovery period.

15. Method according to claim 13, further comprising:
processing predefined battery usage activities and generating a battery usage activity signal in response of detected and processed usage activities;
applying said battery usage activity signal to at least one battery usage timer resulting in that said battery usage timer is started and set to run for a specified duration related to said battery usage activity;
obtaining the battery voltage when no battery usage timer is running and denoting it a valid battery measurement voltage U_{valid}, and
estimating said battery voltage U_{estimated} when a battery usage timer is running, as:
$${U}_{estimated} = {U}_{valid} - {\sum }_{i = 1}^{activities} {N}_{i} {X}_{i}$$
where
Nᵢ is the number of battery usage activities having index i,
Xᵢ is a factor related to said battery usage activity index i, i.e. representing the battery drain caused by said battery usage activity, and
activities represent the number of different kinds of battery usage activities.

16. Method according to claim 15, further comprising determining a linear battery discharge curve being a representation of the relationship between the battery voltage decrease per used ampere-hour capacity and that a presumption to perform said estimation is that the most recent valid battery measurement U_{valid} is less than a threshold value U_{threshold} representing the start of the linear part of the curve.

17. Method according to any of the claim 13, wherein the battery is a QHR battery.

18. An implantable medical device comprising:
at least one device component that performs a battery usage activity related to generation or delivery of in vivo therapy to a patient;
a battery that supplies power to said at least one device component; and
a battery discharge measurement device configured to determine a state of discharge of said battery, said battery discharge measurement device comprising a battery voltage measurement unit adapted to measure and store a battery voltage, a processor unit connected to said battery voltage measurement unit, a battery usage activity detector connected to said processor unit and adapted to detect predefined battery usage activities, and said processor unit being configured to estimate a battery voltage U_{estimated} based on said stored battery voltage and a number of predefined battery usage activities detected by said battery usage activity detector since said battery voltage measurement unit measured said battery voltage.
